(19) United States
(12) Patent Application Publication
(10) Pub. No.: US 2007/0197820 A1
(43) Pub. Date: Aug. 23, 2007

(54) Process for producing urethane (meth)acrylates and new urethane (meth)acrylates

(76) Inventors: Jurgen Van Holen, Gent (DE); Vincent Stone, Schaerbeek (BE); Hugues Van Den Bergen, Drogenbos (BE)

Correspondence Address: WENDEROTH, LIND & PONACK, L.L.P., 2033 K STREET N. W., SUITE 800, WASHINGTON, DC, 20006-1021, US

(21) Appl. No.: 10/591,230

(22) PCT Filed: May 2, 2005
(86) PCT No.: PCT/EP05/04743

§ 371 Date: Aug. 31, 2006

(30) Foreign Application Priority Data
May 17, 2004 (EP) 04011628.7

(51) Int. Cl. C07C 271/08

(52) U.S. Cl. 560/158; 544/222

(57) ABSTRACT

The invention relates to urethane(meth)acrylates and to a process for their production which comprises transesterification of hydroxyalkyl carbamates with an (meth)acrylate of formula [CH₂═CR²⁹—CO—O—]ₜ—R³⁰ wherein R²⁹ is hydrogen or methyl, and R³⁰ represents an alkyl, optionally substituted by hydroxy, which may contain from 1 to 10 ether bridges group, from 1 to 10 —CO— bridges and/or from 1 to 5 —O—CO— bridges, and at least one carbonate of formula (IX) and/or a diester of formula (X) wherein each R³¹, each R³², each R³³, each R³⁴ is, independently, chosen from the group of alkyl and aryl, and R³⁵ is alkylene, alkenylene or arylene.

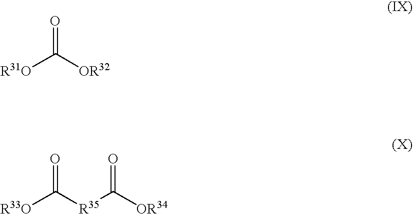

The present invention relates to a process for producing urethane(meth)acrylates and to new urethane(meth)acrylates thereby obtained. The invention also relates to the uses of such compounds, especially in radiation curable compositions.

Radiation curable compositions have found a wide range of applications in numerous fields, for example as coatings, varnishes and paints for protecting and decorating the most diverse substrates such as glass, metals, plastics, paper, as printing varnishes and inks or as adhesives for laminates, and the like. Different processes have been disclosed for the synthesis of urethane (meth)acrylates able to be used in radiation curable compositions. Currently used industrial process for making urethane(meth)acrylates are based on the use of di-isocyanates. These known processes, and especially the di-isocyanate based methods, for making known urethane acrylates or methacrylates use highly toxic and dangerous raw materials, such as low molecular weight di-isocyanates. Storing, handling and processing these toxic raw materials at large scale can only occur with highly expensive secured industrial equipments and safety procedures in an adapted industrial environment.

The present invention aims to find an improved process that overcomes these problems.

The present invention provides a process for producing urethane(meth)acrylates which comprises the reaction of

(a) at least one hydroxyalkyl carbamate of formula (I), (II), (III), (IV), (V), (VI) or (VII)

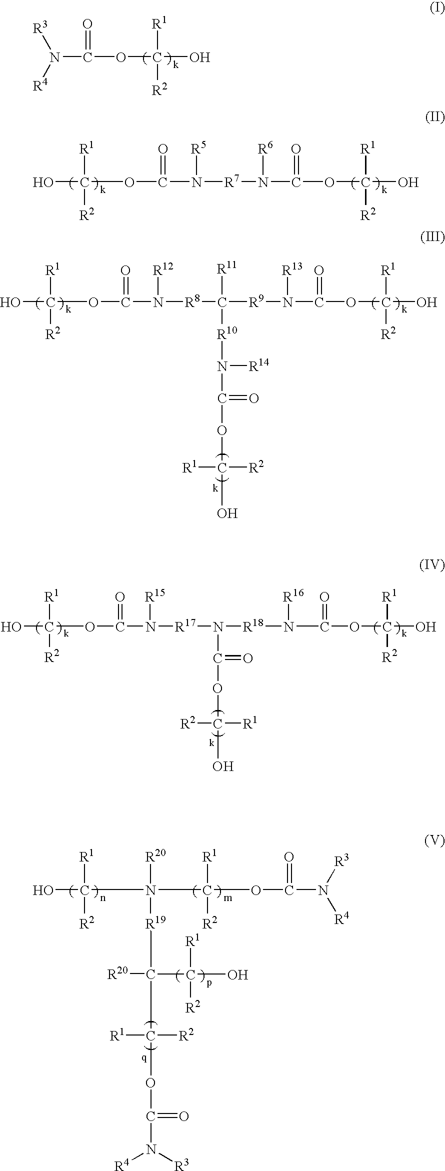

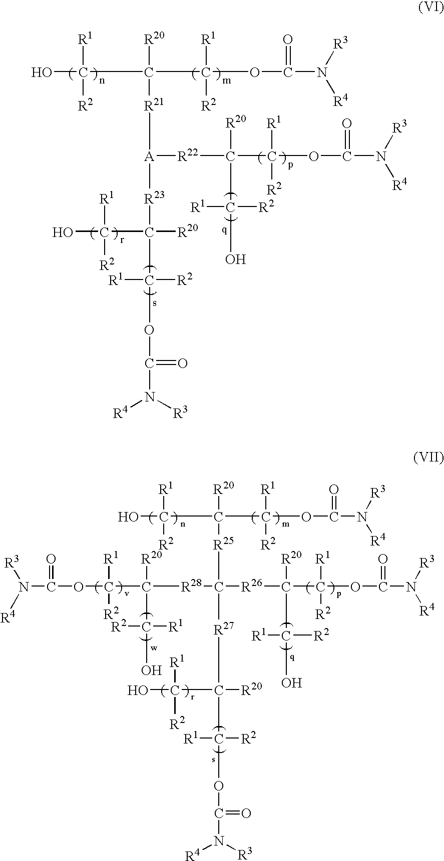

wherein

k≧2

n=0 to 2

m=0 to 2

n+m≧1

p=n or m, q=n or m, r=n or m, v=n or m, v=n or m, w=n or m

(p+q)=(r+s)=(v+w)=(n+m)

each R¹ , each R², each R²⁰ is, independently, chosen from the group of
hydrogen,
halogen,
hydroxy,
alkyl, optionally substituted by hydroxy; halogen; aryl and/or aryl substituted by hydroxy, halogen or alkyl; and optionally containing from 1 to 8 ether bridges,
alkenyl, optionally substituted by hydroxy; halogen; aryl and/or aryl substituted by hydroxy, halogen or alkyl; and optionally containing from 1 to 8 ether bridges,
aryl, optionally substituted by hydroxy; halogen; alkyl; alkyl substituted by hydroxy, halogen and/or aryl; and/or alkyl containing from 1 to 8 ether bridges,
R³ is an alkyl, optionally substituted by hydroxy, tertiary amine and/or aryl, and optionally containing from 1 to 20 ether bridges and/or from 1 to 3 tertiary amine bridges,
R⁴, R⁵, R⁶, R¹², R¹³, R¹⁴, R¹⁵ and R¹⁶ are, independently, chosen from the group of
hydrogen, and
alkyl, optionally substituted by hydroxy, tertiary amine and/or aryl, and optionally containing from 1 to 8 ether bridges and/or from 1 to 3 tertiary amine bridges,
with the proviso that, respectively, R³ and R⁴, R⁵ and R⁶, R¹² and/or R¹³ and/or R¹⁴, R¹⁵ and R¹⁶ may be linked together in order to form a ring,
R⁷, R⁸, R⁹, R¹⁰, R¹⁷ and R¹⁸ are, independently, chosen from alkylene, alkenylene, arylene and aralkylene chains which may contain from 1 to 8 ether bridges and/or from 1 to 3 tertiary amine bridges,
R¹¹ is hydrogen or alkyl;
R¹⁹, R²¹, R²², R²³, R²⁵, R²⁶, R²⁷ and R²⁸, are, independently, chosen from alkylene, alkenylene, arylene and aralkylene chains which may contain from 1 to 20 ether bridges, from 1 to 4 tertiary amine bridges, from 1 to 4 —CO— bridges and/or from 1 to 4 —O—CO— bridges;
A is 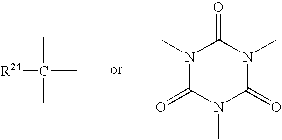
wherein R²⁴ is hydrogen or alkyl;

(b) at least an (meth)acrylate of formula (VIII)
[CH₂═CR²⁹—CO—O]ₜ—R³⁰ (VIII)

wherein R²⁹ is hydrogen or methyl, R³⁰ represents an alkyl, optionally substituted by hydroxy, which may contain from 1 to 10 ether bridges group, from 1 to 10 —O—CO—O— bridges and/or from 1 to 10 —O—CO— bridges, and t≧1; and

(c) at least a carbonate of formula (IX) and/or a diester of formula (X)

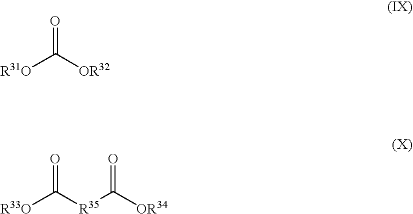

wherein
each R³¹, each R³², each R³³, each R³⁴ is, independently, chosen from the group of alkyl and aryl,
R³⁵ is alkylene, alkenylene or arylene; and

(d) optionally, at least one polyol different from the hydroxyalkyl carbamates (a);

in the presence of at least one transesterification catalyst.

The term “urethane(meth)acrylates” as used in the present invention, is meant to designate products comprising at least one urethane group (—N—CO—O—) and at least one acrylate 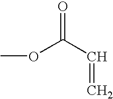
or methacrylate group

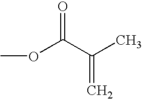

The urethane(meth)acrylates according to the invention can have several repeating units and can therefore also be considered as oligomers or polymers.

The term “alkyl”, as used herein, is defined as including saturated, monovalent hydrocarbon radicals having straight, branched or cyclic moieties or combinations thereof and containing 1 to 50 carbon atoms.

The term “alkenyl” as used herein, is defined as including straight and cyclic, branched and unbranched, unsaturated hydrocarbon radicals having at least one double bond and containing from 2 to 50 carbon atoms; such as ethenyl (=vinyl), 1-methyl-1-ethenyl, 2-methyl-1-propenyl, 1-propenyl, 2-propenyl (=allyl), 1-butenyl, 2-butenyl, 3-butenyl, 4-pentenyl, 1-methyl-4-pentenyl, 3-methyl-1-pentenyl, 1-hexenyl, 2-hexenyl, and the like.

The term “aryl” as used herein, is defined as including an organic radical derived from an aromatic hydrocarbon comprising 1 or more rings by removal of one hydrogen, and containing from 5 to 30 carbon atoms, such as phenyl and naphthyl.

The term “alkoxy”, as used herein, is defined as —O-alkyl groups wherein “alkyl” is as defined above.

The term “alkylene” as used herein, is defined as including saturated, divalent hydrocarbon radicals having straight, branched or cyclic moieties or combinations thereof and containing 1 to 50 carbon atoms.

The term “alkenylene” as used herein, is defined as including unsaturated, divalent hydrocarbon radicals having straight, branched or cyclic moieties or combinations thereof, containing at least one carbon-carbon double bond and containing 1 to 50 carbon atoms.

The term “arylene” as used herein, is defined as including divalent radicals derived from an aromatic hydrocarbon comprising one or more rings by removal of two hydrogen atoms and containing from 5 to 30 carbon atoms.

The term “aralkylene” as used herein, represents a divalent radical comprising a combination of alkylene and arylene moieties.

By alkyl, alkenyl, alkylene, alkenylene, arylene and aralkylene containing an ether bridge is meant an alkyl, alkenyl, alkylene, alkenylene, arylene or aralkylene radical wherein a carbon atom is replaced by an oxygen atom, forming a group such as —C—O—C—.

By alkyl, alkylene, alkenylene, arylene and aralkylene chain containing tertiary amine bridge is meant such radical wherein a tertiary amine group is present between 2 carbon atoms, forming a group of formula —C—NR—C—, wherein R represents an alkyl or aryl group. In that case, R is preferably an alkyl group containing from 1 to 15 carbon atoms.

By alkylene, alkenylene, arylene and aralkylene containing a —CO—O— bridge is meant an alkylene, alkenylene, arylene or aralkylene radical wherein a 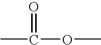
group is present between 2 carbon atoms, forming a group of formula

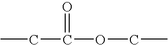

By alkylene, alkenylene, arylene and aralkylene containing a —CO— bridge is meant an alkylene, alkenylene, arylene or aralkylene radical wherein a 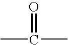
group is present between 2 carbon atoms.

By alkyl a —O—CO—O— bridge is meant an alkyl wherein a 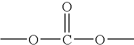
group is present between 2 carbon atoms, forming a group of formula

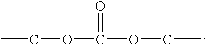

The hydroxyalkyl carbamates of formula (I), (II), (III), (IV), (V), (VI) and (VII) used in the process according to the invention may be obtained by any method suitable therefore.

Hydroxyalkyl carbamates of formula (I), (II), (III) and (IV) are preferably obtained by reacting amines of, respectively, formula (IX), (X), (XI) and (XII) 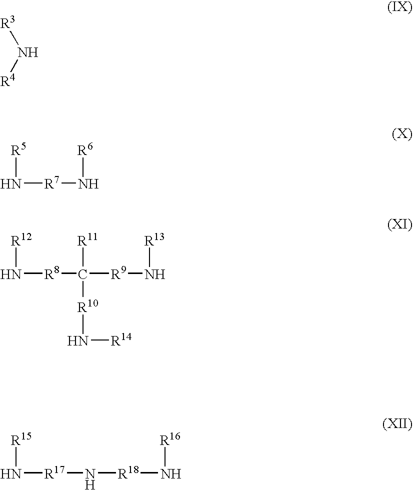
wherein R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸, are defined as here above, with a cyclic carbonate of formula (XIII) 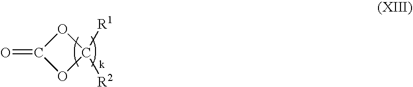
wherein R¹, R² and k are defined as here above.
Hydroxyalkyl carbamates of formula (V), (VI) and (VII) are preferably obtained by reacting an amine of formula (IX) 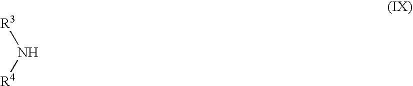
wherein R³ and R⁴ are defined as here above, with, respectively, a cyclic carbonate of formula (XIV), (XV) and (XVI) 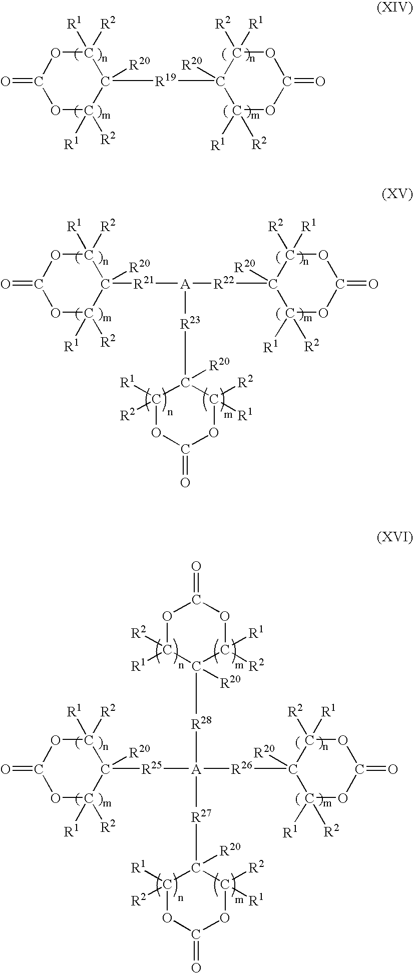
wherein R¹, R², R¹⁹, R²⁰, R²¹, R²², R²³, R²⁴, R²⁵, R²⁶, R²⁷, R²⁸, A, n and m are defined as here above.

The preparation of the hydroxyalkyl carbamates by the above mentioned processes is exothermic. Therefore, the reaction temperature is preferably controlled in order to avoid side reactions which could give toxic by-products or which could lead to products which form toxic by-products further in the process according to the invention. During the preparation processes, the temperature is in general maintained below 100° C., preferably between 40 and 90° C. and most preferably between 50 and 80° C. When a primary amine is used, the reaction may be so exothermic that it is difficult to maintain the reaction mixture within the above temperature range. It is then preferred to add the cyclic carbonate to the amine in order to have a better control of the reaction temperature.

During the preparation of the hydroxyalkyl carbamates, the reaction mixture is preferably sparged with nitrogen. This sparging with nitrogen helps to avoid moisture that can lead to hydrolysis of the cyclic carbonate catalyzed by the amine. Sparging with nitrogen can also help in reducing the color of the hydroxyalkyl carbamate, especially when readily oxidized amines are used such as secondary amines or amines with ether groups. Alternatively or in addition to the sparging with nitrogen, an antioxidant may be used during the preparation of the hydroxyalkyl carbamates. Preferred antioxidants are aromatic phosphites, most preferred are triphenylphosphite or trisnonylphenylphosphite. The amount of these antioxidants is preferably at least 300 ppm w/w based on the quantity of hydroxyalkyl carbamate formed.

In general, this amount does not exceed 5000 ppm, preferably not 2000 ppm w/w based on the quantity of hydroxyalkyl carbamate formed.

During the preparation of the hydroxyalkyl carbamates, the equivalent ratio amine to cyclic carbonate is preferably of 1.01 to 1.1 and most preferably, of 1 to 1.05. The equivalent ratio amine to cyclic carbonate is meant to designate the number of primary and secondary amine groups of the amine of formula (IX), (X), (XI) or (XII) per number of cyclic carbonate groups present in the cyclic carbonates of formula (XIII), (XIV), (XV) or (XVI) used. Especially when the boiling point of the amine is lower than 100° C. so that it can be stripped off the reactor under vacuum below this temperature, it is preferred working with an amine excess to so as speeding up the reaction. This is especially true for secondary amines with which reaction times are typically longer due to steric hindrance around the opening proton.

During the preparation of the hydroxyalkyl carbamates, a catalyst can be used. Secondary, higher molecular weight primary or hindered primary amines may lead to very slow ring opening reactions, especially when substituent(s) lie on the cyclic carbonate rings. When these amines are used, it is preferred to use one or a combination of the known catalysts for this opening reaction, such as strongly basic amines (such as diazabicyclooctane, tetramethylguanidine), strongly basic quaternary ammonium compounds (such as alkyl(C16-C22)benzyltrimethyl ammonium hydroxide or carbonate and tetrabutylammonium hydroxide or carbonate), supranucleophilic catalysts (such as 4-pyrrolidinepyridine, poly(N,N-dialkylaminopyridine, dimethylaminopyridine), base anions whose conjugate acid has a pKa of about 11 or more (such as tert-butoxide).

While it is preferred not to use any solvent for the preparation reaction of the hydroxyalkyl carbamates, the latter may be used, for example, in order to improve the compatibility of a reagent or a catalyst such as the reaction mixture is homogeneous throughout the reaction. According to a variant of the process according to the invention, a carbonate of formula (IX) and/or a diester of formula (X) is used as solvent during the preparation of the hydroxyalkyl carbamates.

In the hydroxyalkyl carbamates of formula (I), (II), (III), (IV) and in the cyclic carbonates of formula (XIII) used in the process according to the invention k is preferably at most 3, most preferably k is 2.

In the hydroxyalkyl carbamates of formula (V), (VI) and (VII) and in the cyclic carbonates of formula (XIV), (XV) and (XVI) used in the process according to the invention n, m, p, q, r, s, v and w are, independently, preferably 0 or 1.

In the hydroxyalkyl carbamates of formula (V), (VI) and (VII) and in the cyclic carbonates of formula (XIV), (XV) and (XVI) used in the process according to the invention n+m, p+q, r+s, v+w, are preferably 1.

In the hydroxyalkyl carbamates of formula (I), (II), (III), (IV), (V), (VI) and (VII) and in the cyclic carbonates of formula (XIII), (XIV), (XV) and (XVI) used in the process according to the invention each R¹ and each R² is, independently, preferably chosen from the group of hydrogen; alkyl comprising from 1 to 6 carbon atoms, optionally substituted by hydroxy or halogen; and alkenyl comprising from 1 to 6 carbon atoms; both optionally containing from 1 to 3 ether bridges.

In the hydroxyalkyl carbamates of formula (I), (II), (III), (IV), (V), (VI) and (VII) and in the cyclic carbonates of formula (XIII), (XIV), (XV) and (XVI) used in the process according to the invention, preferably, all but one of the R¹ substituents are hydrogen. In these preferred hydroxyalkyl carbamates, all but one of the R² substituents are preferably hydrogen. Most preferably, in these hydroxyalkyl carbamates, all R² substituents are hydrogen.

Particularly preferred hydroxyalkyl carbamates of formula (I), (II), (III) and (IV) and particularly preferred cyclic carbonates of formula (XIII) are those wherein one of the R¹ substituents is chosen from the group of hydrogen, methyl, ethyl, hydroxymethyl, chloromethyl, allyloxymethyl, and wherein the R² substituent present on the same carbon atom as this R¹ substituent is chosen from hydrogen and methyl, all other R¹ and all other R² substituents being hydrogen.

Particularly preferred hydroxyalkyl carbamates of formula (V), (VI) and (VII) and particularly preferred cyclic carbonates of formula (XIV), (XV) and (XVI) are those wherein each R¹ and each R² is hydrogen. In the hydroxyalkyl carbamates of formula (V), (VI) and (VII) and in the cyclic carbonates of formula (XIV), (XV) and (XVI) used in the process according to the invention each R²⁰ is preferably hydrogen.

In the hydroxyalkyl carbamates of formula (I), (V), (VI) and (VII) and in the amines of formula (IX) used in the process according to the invention R³ is preferably an alkyl, optionally substituted by hydroxy, tertiary amine and/or aryl, and optionally containing from 1 to 20 ether bridges. Most preferably, R³ is chosen from the group of alkyl comprising up to 10 carbon atoms, optionally substituted by one hydroxy or tertiary amine and/or optionally containing one or two ether bridges. Non-limiting examples are R³ substituents chosen from the group of n-propyl, isopropyl, n-butyl, isobutyl, sec-butyl, tert-butyl, n-hexyl, n-octyl, 2-ethylhexyl, isononyl, cyclopentyl, cyclohexyl, 2-methylcyclohexyl, N,N-(di-tert-butyl)ethyl, benzyl, 2-(2-hydroxyethoxy)ethyl, 5-hydroxypentyl, 2-hydroxyethyl, 2-hydroxypropyl, 3-hydroxypropyl, 3-(diethylamino)propyl, 2-(diethylamino)ethyl, 1-methyl-4-(diethylamino)butyl, 2-((di-tert-butyl)amino)ethyl, 3-(dimethylamino)propyl, 2-methoxyethyl, 2-ethoxyethyl, 3-methoxypropyl, 2-methoxyisopropyl, 3-ethoxypropyl, 3-isopropoxypropyl, 3-(2-methoxyethoxy)propyl, 3-(2-ethylhexyloxy)propyl, CH₃O(CH₂CH₂O)₆—(CH₂CHR—O)₁₀—CH₂—CH(CH₃)— wherein R is H or CH₃ in a proportion of 1:9, ethyl, methyl, 1,2-dimethylpropyl.

In the hydroxyalkyl carbamates of formula (I), (V), (VI) and (VII) and in the amines of formula (IX) used in the process according to the invention R⁴ is preferably chosen from the group of hydrogen and alkyl, optionally substituted by hydroxy, tertiary amine or aryl, and optionally containing from 1 to 8 ether bridges. Most preferably, R⁴ is chosen from the group of hydrogen and alkyl comprising up to 10 carbon atoms, optionally substituted by one hydroxy or tertiary amine and/or optionally containing one or two ether bridges. Non-limiting examples are R⁴ substituents chosen from the group of hydrogen, ethyl, n-propyl, isopropyl, n-hexyl, methyl, tert-butyl, n-butyl, isobutyl, n-octyl, 2-ethylhexyl, 1,2-dimethylpropyl, cyclohexyl, 2-hydroxyethyl, 2-hydroxyisopropyl, 3-hydroxypropyl, 2-methoxyethyl, 3-(dimethylamino)propyl.

In the hydroxyalkyl carbamates of formula (I), (V), (VI) and (VII) and in the amines of formula (IX) used in the process according to the invention R⁴ is more preferably hydrogen and R³ is as defined here above, more specifically R³ is an alkyl comprising at least 3 carbon atoms and substituted by at least one hydroxy and optionally containing one or two ether bridges. Particularly preferred R⁴ is hydrogen and R³ is 2-(2-hydroxyethoxy)ethyl.

In the hydroxyalkyl carbamates of formula (II), (III), (IV) and in the amines of formula (X), (XI) and (XII) used in the process according to the invention R⁵, R⁶, R¹², R¹³, R¹⁴, R¹⁵ and R¹⁶ are preferably, independently, chosen from the group of hydrogen and alkyl comprising up to 10 carbon atoms, most preferably up to 6 carbon atoms.

In the hydroxyalkyl carbamates of formula (I), (II), (III), (IV), (V), (VI) and (VII) and in the amines of formula (IX), (X), (XI) and (XII) used in the process according to the invention R³ and R⁴, R⁵ and R⁶, R¹² and/or R¹³ and/or R¹⁴, R¹⁵ and R¹⁶, respectively, may be linked together in order to form a ring. In this case, these substituents are preferably linked so that they form an alkylene chain comprising from 2 to 7 carbon atoms, and optionally containing 1 or 2 ether bridges. In case of R³ and R⁴, this alkylene chain is preferably such that a 5 to 7-membered ring is formed, for example a pyrolidine ring, a piperidine ring or a morpholine ring, which may further be substituted by alkyl groups. In case of R⁵ and R⁶, this alkylene chain is preferably such that a 5 to 7-membered ring is formed, for example piperazine, which may further be substituted by alkyl groups.

In the hydroxyalkyl carbamates of formula (II) and in the amines of formula (X) used in the process according to the invention R⁷ is preferably chosen from the group of alkylene and aralkylene chains, containing up to 20 carbon atoms and which may contain from 1 to 8 ether bridges and/or from 1 to 3 tertiary amine bridges. Most preferably, R⁷ is chosen from the group of ethylene, 1,2-propylene, trimethylene, hexamethylene, 2,2-dimethylpropylene, 1-methyltrimethylene, 1,2,3-trimethyltetramethylene, 2-methyl-pentamethylene, 2,2,4-(or 2,4,4-)trimethylhexamethylene, metaxylylene, 3,5,5-trimethylcyclohexyl-1-ene-3-methylene, bis(cyclohexyl-4-ene)methane, bis(4-methylcyclohexyl-3-ene)methane, cyclohexyl-1,3-ene, cyclohexyl-1,4-ene, 1,4-bis(propoxyl-3-ene)butane, N,N-bis(trimethylene)methylamine, 3,6-dioxaoctylene, 3,8-dioxadodecylene, 4,7,10-trioxatridecylene, poly(oxytetramethylene), poly(oxypropylene) with 2 to 15 1,2-propylene oxide units, poly(oxypropylene-co-oxyethylene) with 2 to 15 propylene oxide and 2 to 15 ethylene oxide units.

Especially preferred is 2,2-dimethylpropylene.

In the hydroxyalkyl carbamates of formula (III) and in the amines of formula (XI) used in the process according to the invention R⁸, R⁹, R¹⁰ are preferably, independently, chosen from the group of alkylene, optionally containing from 1 to 8 ether bridges. Most preferably R⁸, R⁹, R¹⁰ are chosen from alkylene comprising up to 15 carbon atoms and containing up to 5 ether bridges.

In the hydroxyalkyl carbamates of formula (IV) and in the amines of formula (XII) used in the process according to the invention R¹⁷ and R¹⁸ are preferably, independently, chosen from the group of alkylene.

Most preferably R¹⁷and R¹⁸ are chosen from alkylene comprising up to 6 carbon atoms.

In the hydroxyalkyl carbamates of formula (III) and in the amines of formula (XI) used in the process according to the invention R¹¹ is preferably hydrogen or an alkyl containing from 1 to 4 carbon atoms.

In the hydroxyalkyl carbamates of formula (V) and in the cyclic carbonates of formula (XIV) used in the process according to the invention R¹⁹ is preferably chosen from alkylene and aralkylene chains which may contain from 1 to 20 ether bridges. Prefered are alkylene and aralkylene chains comprising at least 2 ether bridges.

In the hydroxyalkyl carbamates of formula (VI) and (VII) and in the cyclic carbonates of formula (XV) and (XVI) used in the process according to the invention R²¹, R²², R²³, R²⁵, R²⁶, R²⁷and R²⁸, are preferably, independently, chosen from alkylene and aralkylene chains which may contain from 1 to 3 ether bridges.

In the hydroxyalkyl carbamates of formula (VI) and in the cyclic carbonates of formula (XV) used in the process according to the invention R²⁴ is preferably hydrogen or alkyl comprising form 1 to 4 carbon atoms.

Cyclic carbonates such as used in the process according to the invention are known in the art or can easily be prepared by known methods. Cyclic carbonates of formula (XIII) wherein k=2 and cyclic carbonates of formulas (XIV), (XV) and (XVI) wherein n+m, p+q, r+s or v+w are equal to 1 can easily be prepared by the reaction of the corresponding epoxides with carbon dioxide. Typical reaction conditions are described in Kihara, N., Hara, N., Endo, T., J. Org. Chem., 1993, 58, 6198., J. Org. Chem., 1993, 58, 6198-6202. Some of the catalysts described hereabove for the preparation of the hydroxyalkyl carbamates by opening of the cyclic carbonate with the amine, are also known as catalysts for the reaction between carbon dioxide and an epoxide group. Cyclic carbonates of formula (XIII) wherein k=2 and cyclic carbonates of formulas (XIV), (XV) and (XVI) wherein n+m, p+q, r+s or v+w are equal to 1 can easily be prepared by the reaction of the corresponding epoxides with carbon dioxide. In this case, it is preferred to choose a catalyst active for both the formation of the cyclic carbonate group(s) and their opening by the amine.

Cyclic carbonates of formula (XIII) wherein k=3 can be prepared by transesterification of propane-1,3-diols with dialkyl carbonates such as described for example in Hu, B., Zhuo, R. X., Fan, C. L., Polym. Adv. Technol., 1998, 9, 145. Cyclic carbonates of formulas (XIV), (XV) and (XVI) wherein n+m, p+q, r+s or v+w are equal to 2 can be prepared by transesterification of polyols containing several propane-1,3-diols groups such as ditrimethylolpropane with dialkyl carbonates. They can also be obtained by reacting 6-membered cyclic carbonates containing one functional group such as OH group in 5-ethyl-5-hydroxymethyl-1,3-dioxan-2-one with coupling agents such as multifunctional chloroformates.

Cyclic carbonates of formula (XIII) wherein k>3 and cyclic carbonates of formulas (XIV), (XV) and (XVI) wherein n+m, p+q, r+s or v+w are >2 can be prepared according to the same transesterification reaction pathways as that leading to the lower-membered rings (Matsuo, J. et al., J. Polym. Sci. A: Polym. Chem., 1997, 35, 1375).

Cyclic carbonates of formula (XIII) which are particularly useful in the process according to the invention are 1,3-dioxolan-2-one (ethylene carbonate), 4-methyl-1,3-dioxolan-2-one(propylene carbonate), 4-ethyl-1,3-dioxolan-2-one(butylene carbonate), 4-hydroxymethyl-1,3-dioxolan-2-one (glycerine carbonate), 4-chloromethyl-1,3-dioxolan-2-one, 4-allyloxymethyl-1,3-dioxolan-2-one, 5,5-dimethyl-1,3-dioxan-2-one, (neopentylglycol carbonate).

Cyclic carbonates of formulas (XIV), (XV) and (XVI) which are particularly useful in the process according to the invention are those obtained from the reaction of carbon dioxide with polyepoxide compounds, such as the polyglycidyl ethers of aliphatic or aromatic polyols, such as, for example, 1,4-butanediol, neopentylglycol, cyclohexanedimethanol, diethyleneglycol, polyethyleneglycol, dipropyleneglycol, polypropyleneglycol, 2,2,4-trimethyl-1,3-pentanediol, 1,6-hexanediol, trimethylolpropane, trimethylolethane, glycerol, 4,4′-isopropylidenediphenol, 1,1,1-tris(4-hydroxyphenyl)ethane, hydroquinone, 4,4′-bisphenol, 2,2′-bisphenol, 4,4′-dihydroxybenzophenone, 1,5-dihydroxynaphthalene, resorcinol. Preferred are the polyglycidyl ethers of aliphatic polyols, and most preferred the diglycidyl ether of polypropyleneglycol containing from 2 to 15 1,2-propylene oxide units.

Amines of formula (IX), (X), (XI) and (XII) are known in the art. Amines of formula (IX) which are particularly useful in the process according to the invention are n-propylamine, isopropylamine, n-butylamine, isobutylamine, sec-butylamine, tert-butylamine, 3-methylbutylamine, n-hexylamine, n-octylamine, 2-ethylhexylamine, isononylamine, cyclopentylamine, cyclohexylamine, 2-methylcyclohexylamine, N,N-(di-tert-butyl)ethyleneamine, benzylamine, 2-(2-aminoethoxy)ethanol, 5-aminopentanol, ethanolamine, 1-aminopropan-2-ol, 3-amino-1-propanol, 3-(diethylamino)propylamine, 2-(diethylamino)ethylamine, 1-methyl-4-(diethylamino)butylamine, 2,2-(di-tert-butylamino)ethylamine, 3-(dimethylamino)propylamine, 2-methoxyethylamine, 2-ethoxyethylamine, 3-methoxypropylamine, 1-methoxyisopropylamine, 3-ethoxypropylamine, 3-isopropoxypropylamine, 3-(2-methoxyethoxy)propylamine, 3-(2-ethylhexyloxy)propylamine, α-oxymethyl-ω-(2-propylamino)-poly(oxypropylene-co-oxyethylene) with an average number of 1,2-propylene oxide units of 9 and an average number of ethylene oxide units of 7, also known as Jeffamine® M-600 (manufactured by Hunstman), diethylamine, di-n-propylamine, diisopropylamine, di-n-hexylamine, N-methylbutylamine, N-ethylbutylamine, di-n-butylamine, diisobutylamine, di-n-octylamine, bis(2-ethylhexyl)amine, N-ethyl-1,2-dimethylpropylamine, dicyclohexylamine, cyclohexylmethylamine, cyclohexylethylamine, N-methylbenzylamine, 2-methylaminoethanol, 2-ethylaminoethanol, 2-butylaminoethanol, diethanolamine, diisopropanolamine, 3-(2-hydroxyethyl)aminopropanol, bis(2-methoxyethyl)amine, bis(3-dimethylaminopropyl)amine, pyrolidine, piperidine, morpholine, 2,6-dimethylmorpholine.

Amines of formula (X) which are particularly useful in the process according to the invention are ethylenediamine, 1,2-propylenediamine, trimethylenediamine, hexamethylenediamine, 2,2-dimethylpropane-1,3-diamine, 1-methyl-1,3-propanediamine, 1,2,3-trimethyl-1,4-butanediamine, 2-methyl-1,5 diaminopentane, 2,2,4-(or 2,4,4-)trimethylhexamethylene diamine, metaxylylenediamine, 1-amino-3-aminomethyl-3,5,5 trimethylcyclohexane (isophorone diamine), bis-(4-aminocyclohexyl)-methane, bis-(4-amino-3-methyl-cyclohexyl)-methane, 1,3-cyclohexanediamine, 1,4-cyclohexanediamine, 1,4-Bis(3-aminopropoxy)butane diamine, N,N-bis(3-aminopropyl)methylamine, triethyleneglycol diamine, 3,3′-(butane-1,4-diylbis(oxy))bispropaneamine, 4,7,10-trioxatridecan-1,13-diamine, α-amino-ω-(4-butylamino)-poly(oxytetramethylene), α-amino-Ω-(2-propylamino)-poly(oxypropylene) with an average number of 1,2-propylene oxide units of 2.6, also known as Jeffamine® D-230 (manufactured by Hunstman), α-amino-Ω-(2-propylamino)-poly(oxypropylene) with an average number of 1,2-propylene oxide units of 5.6, also known as Jeffamine® D-400 (manufactured by Hunstman), α-amino-ω-(2-propylamino)-poly(oxypropylene-co-oxyethylene) with an average number of 1,2 propylene oxide units of 2.5 and ethylene oxide units of 8.5, also known as Jeffamine® ED-600 (manufactured by Hunstman), N,N′-dimethyl-1,3-propanediamine, N,N′-di-tert-butyl-ethanediamine, N,N′-dimethylhexyl-1,6-diamine, piperazine, 2,5-dimethylpiperazine.

Amines of formula (XI) that are particularly useful in the process according to the invention is propoxylated trimethylopropane triamine with an average number of number of 1,2-propylene oxide units of 5.3, also known as Jeffamine® T-403 (manufactured by Hunstman).

Amines of formula (XII) that are particularly useful in the process according to the invention are N,N-dimethyldipropylenetriamine, bis(hexamethylene)triamine.

In the (meth)acrylates of formula (VIII) used in the process according to the invention, t is preferably from 1 to 6. Most prefered are the (meth)acrylates wherein t is 1.

When the amount of carbonate of formula (IX) and/or diester of formula (X) used in the process is such that a stoechiometric excess of hydroxy groups, originating from the hydroxyalkyl carbonate (a) and from the polyol (d), when present, is present in the reaction mixture, the (meth)acrylate of formula (VIII) is preferably a (meth)acrylate wherein R³⁰ is an alkyl comprising from 1 to 6 carbon atoms, most preferably, methyl, ethyl or n-butyl.

When the amount of carbonate of formula (IX) and/or diester of formula (X) used in the process is such that a stoechiometric excess of carbonate or ester groups relative to the hydroxy groups is present in the reaction mixture, the (meth)acrylate of formula (VIII) is preferably a (meth)acrylate wherein R³⁰ is an alkyl substituted by at least one hydroxy group, and which may contain from 1 to 10 ether bridges group, from 1 to 10 —O—CO—O— bridges or from 1 to 10 —O—CO— bridges. In that case R³⁰ is more preferably an alkyl comprising 1 to 6 carbon atoms and one hydroxy group.

Prefered (meth)acrylates of formula (VIII) are methylacrylate, ethylacrylate, methylmethacrylate, ethylmethacrylate, hydroxyethylacrylate, hydroxyethylmethacrylate, hydroxypropylacrylate, hydroxypropylmethacrylate, hydroxybutylacrylate, cyclohexanedimethanolmonoacrylate, pentaerythritol triacrylate, the reaction product of hydroxyethyl(meth)acrylate with 1 to 10 caprolactone molecules (such as the products commercialized under the name of Tone M-100, Tone M-101 and Tone M-201 by Dow), the reaction product of hydroxyethyl(meth)acrylate with 1 to 10 molecules containing a 6-membered cyclic carbonate group.

In the carbonates of formula (IX) used in the process according to the invention R³¹ and R³² are preferably chosen from the group of alkyl comprising from 1 to 4 carbon atoms and from phenyl. Preferred carbonates are dimethylcarbonate, diethylcarbonate and diphenylcarbonate.

In the diesters of formula (X) used in the process according to the invention R³³ and R³⁴ are preferably chosen from the group of alkyl comprising from 1 to 6 carbon atoms and from phenyl. Most preferred R³³ and R³⁴ are methyl.

In the diesters of formula (X) R³⁵ is preferably alkylene or alkenylene comprising from 1 to 10 carbon atoms or phenylene.

Preferred diesters are dimethyladipate, dimethylglutarate, dimethylitaconate, dimethylsuccinate, dimethylmalonate, dimethylsuberate, dimethylsebacate, dimethylphthalate, dimethylterephthalate and dimethylisophthalate.

The polyol (d) different from the hydroxyalkyl carbamates (a) optionally used in the process of the invention can be any polyol containing at least 2 hydroxy groups, including molecules having a molecular weight up to 4000.

The polyol preferably responds to formula B—(OH)ₓ wherein x is an integer from 1 to 6 and B represents an alkyl or alkenyl optionally containing from 1 to 100 ether bridges, —CO—O— bridges, —CO— bridges and/or —O—CO—O— bridges and/or containing one or more (preferably not more than 6) —COOH, —SO₃H and/or —PO₄H groups. Examples of such polyols are ethylene glycol, propyleneglycol, 1,4-butanediol, 1,5-pentanediol, neopentylglycol, 1,6-hexanediol, diethyleneglycol, triethyleneglycol, dipropyleneglycol, tripropyleneglycol, cyclohexanedimethanol, dimethylolpropionic acid, trimethylolpropane, pentaerythritol and macrodiols such as polyetherdiols, polyesterdiols, polycarbonatediols, polyestercarbonatediols, polybutadienediol, acrylic diols. Preferred are macrodiols having a molecular weight from 100 to 4000.

While it is generally not necessary to use any solvent for the transesterification reaction, the latter may be used, for example, in order to improve the compatibility of a reagent or a catalyst so that the reaction mixture is homogeneous throughout the reaction. Inert solvents with form an azeotrope with the alcohol generated during the transesterification reaction are preferred. More prefered are cyclohexane, toluene, acetone, dioxane or their mixtures.

The catalyst used in the transesterification reaction can be an organotin catalyst. The organotin catalyst is preferably chosen from dibutyltin oxide, monobutyltin oxide, monobutyltindihydroxychloride, n-butyl tin tris(2-ethylhexanoate), dibutyltindilaurate, dioctyltindilaurate, dibutyltinmaleate, dibutyltindiacetate, dibutyltindiisooctoate, dibutyltincarboxylate, dimethyltindichloride, and their mixtures; it is most preferably dibutyltindilaurate and/or dioctyltindilaurate.

The catalyst used in the transesterification reaction can be an organotitanate catalyst. The organotitanate catalyst is preferably chosen from alkyltitanates, more particularly from tetraalkyltitanates, wherein each alkyl, independently, comprises from 2 to 8 carbon atoms. More preferred are isopropyltitanate, n-butyltitanate, ethyltitanate, n-propyltitanate, 2-ethylhexyltitanate and their mixtures. Particularly preferred are isopropyltitanate, n-butyltitanate and their mixtures.

The catalyst used in the transesterification reaction can be an organozirconate catalyst. The latter is preferably chosen from zirconium acetate, zirconium acetylacetonate, zirconium hexafluoroacetylacetonate, zirconium trifluoroacetylacetonate, zirconium propionate, zirconium 2-ethylhexanoate, zirconium t-butoxide, zirconium n-butoxide, zirconium ethoxide, zirconium n-propoxide, zirconium isopropoxide, zirconium chloride, zirconium bromide, zirconium fluoride, zirconium iodide, zirconium oxychloride, zirconium hydrochloride, zirconium methacrylate and their mixtures; it is most preferably zirconium acetylacetonate and/or zirconium n-butoxide.

The organozirconate catalyst is preferably chosen from alkylzirconates, more particularly from tetraalkylzirconates, wherein each alkyl, independently, comprises from 2 to 8 carbon atoms and from zirconium 1,3-diketone chelates, and their mixtures.

As described in FR2747675 (Elf Atochem SA), any of these zirconium catalysts can be doped with β-diketones (e.g. acetylacetone), esters of ketonic acid (e.g. ethylacetoacetate) or β-hydroxydiketones (e.g. hydroxybenzophenone).

The organozirconium catalyst is most preferably chosen from alkylzirconates, especially zirconium n-butoxide, in combination with zirconium acetylacetonate and/or a β-diketone, especially acetylacetone.

The amount of catalyst used in the transesterification reaction according to the invention, is preferably such that the weight ratio of catalyst to the generated urethane(meth)acrylate is at least 0.001, preferably at least 0.005. The weight ratio does preferably not exceed 0.2, most preferably not 0.1. A too low concentration in catalyst could lead to unacceptable yields of the desired urethane(meth)acrylate; higher concentrations could lead to the formation of undesirable side products and/or could influence the properties of the final product, necessitating or complicating its removal.

Although not absolutely required, residual catalyst may be removed from the reaction mixture obtained after the transesterification reaction. Organotin catalysts may be removed by one of the methods known in the art. Methods to get rid of tin catalysts from organic media relies either on adding a reagent that makes them insoluble in the matrix or washing methods by hydrolytic treatments. Organotitanate and organozirconate catalysts can be easily removed by adding any substance (polyol, water, . . . ) able to react with this catalyst and forming an insoluble precipitate that can be removed from the reaction mixture. Organozirconate catalysts can be removed by washing with water.

The amount of (meth)acrylate of formula (VIII) used in the transesterification reaction according to the invention, is preferably such that the equivalent ratio of (meth)acrylate to hydroxyalkyl carbamate is at least 0.01. By equivalent ratio of (meth)acrylate to hydroxyalkyl carbamate is understood the number of equivalents of (meth)acrylate of formula (VIII) to the number of equivalents of hydroxy groups present in the hydroxyalkyl carbamate. The equivalent ratio is preferably at least 0.1, more preferably at least 0.2. The equivalent ratio does preferably not exceed 5, most preferably not 1.

A part of the excess (meth)acrylate is preferably continuously distilled as an azeotrope with the corresponding alcohol formed during the transesterification reaction. The remaining part of the excess may be removed at the end of the transesterification reaction by concentration/stripping under vacuum, preferably under injection of air in order to prevent polymerization. This remaining part of the excess is preferably removed after removal of the residual catalyst.

The amount of dicarbonate of formula (IX) and/or diester of formula (X) used in the transesterification reaction according to the invention, is preferably such that the equivalent ratio of carbonate and/or diester to hydroxyalkyl carbamate is at least 0.05. By equivalent ratio of carbonate and/or diester to hydroxyalkyl carbamate is understood the number of equivalents of carbonate and/or diester to the number of equivalents of hydroxy groups present in the hydroxyalkyl carbamate. The equivalent ratio is preferably at least 0.1, more preferably at least 0.5. The equivalent ratio does preferably not exceed 20, most preferably not 10.

The amount of polyol (d) optionally used in the transesterification reaction according to the invention, is preferably such that the equivalent ratio of polyol to hydroxyalkyl carbamate is from 0 to 50. By equivalent ratio of polyol to hydroxyalkyl carbamate is understood the number of equivalents of polyol to the number of equivalents of hydroxy groups present in the hydroxyalkyl carbamate. The equivalent ratio is preferably at least 0.05, more preferably at least 0.2. The equivalent ratio does preferably not exceed 50, most preferably not 20.

The transesterification reaction in the process according to the invention is preferably effectuated at temperatures of at least 40° C., more preferably of at least 50° C., and most preferably of at least 60° C.

The transesterification reaction is generally effectuated at a temperature of at most 120° C., more preferably of at most 110° C.

The maintenance of the temperature during the transesterification reaction can be done by any means known therefore. The maintenance of the temperature is preferably achieved by distilling off, in general under reduced pressure, the alcohol generated during the reaction, preferably as an azeotrope with a solvent. When present, the remaining part of the solvent may be removed at the end of the transesterification reaction, for example by concentration/stripping under vacuum, preferably under injection of air. This remaining part of solvent is preferably removed after removal of residual catalyst.

In the process according to the invention, the different reagents can be added to the reaction mixture from the start. Alternatively, one or more reagents, especially the (meth)acrylate of formula (VIII), are added some time after the others. A combination of both methods can also be used.

The transesterification reaction is usually conducted in the presence of at least one polymerization inhibitor. By polymerization inhibitor is understood an additive which slows or inhibits the polymerization of the reactives and/or the formed products. Preferred polymerization inhibitors are chosen from phenothiazine, triphenylstibine, triphenylphosphine, quinones (such as para-benzoquinone), nitrones, nitro- and nitroso compounds (such as nitrobenzene), stable radicals (such as 2,2,6,6-tetramethyl-1-piperidinyloxy and diphenylpicrylhydrazil), 2,6-di-tert-butyl-4-methylphenol (BHT), 2,6-dimethylphenol, 2,2′-methylenebis(4-methyl-6-(1-methyl-cyclohexyl) phenol), 2,2′-methylenebis(4-methyl-6-tert-butylphenol), 2,2′-methylenebis(4-ethyl-6-tert-butylphenol), 2,4-dimethyl-6-(1-methylpentadecyl)-phenol, alpha-tocopherol (vitamin E) and their mixtures.

The amount of polymerization inhibitor present during the transesterification reaction is in general at least 100 ppm w/w, preferably at least 300 ppm w/w based on the quantity of urethane(meth)acrylate formed.

The amount of polymerization inhibitor preferably does not exceed 5000 ppm w/w, most preferably not 3000 ppm w/w based on the quantity of final product formed.

The process according to the invention permits to obtain urethane(meth)acrylates of high purity, containing no or less toxic side products than the processes disclosed in the prior art, even:without the use of extra purification procedures. The process according to the invention permits to obtain products containing no residual isocyanates. The process according to the invention also presents the advantage that cheap and safe raw materials can be used. The process involving nor toxic raw materials nor highly exothermic reactions leading to atmospheric pollution with toxic materials, standard industrial equipments and safety procedures can be used at larger scale.

The process according to the invention permits to obtain new urethane (meth)acrylates not known in the prior art, most of which are not obtainable by the current commercial processes such as the isocyanate process.

The present invention also relates to new urethane(meth)acrylates that can be obtained with the process according to the present invention.

These urethane(meth)acrylates have in general a much lower viscosity as compared to existing urethane(meth)acrylates having similar molecular weights and same acrylate functionality.

The new low viscous urethane(meth)acrylates allow reducing significantly the amount of reactive diluents needed to make the radiation-curable formulations. This allows maximizing properties specifically brought by (meth)acrylates with urethane residues (adhesion, abrasion resistance . . . ) and makes possible the use of urethane(meth)acrylates to applications where the viscosity of the formulation should be very low, such as spray coatings. Besides lowering the overall irritancy and smell of the formulation, reducing the amount of reactive diluents also allows reducing the well-known detrimental effect(s) the latter may have on specific coatings and inks properties. For example, reducing the amount of reactive diluents in UV parquet topcoats formulations is indeed well-known to improve significantly their abrasion resistance. In radiation-curable flexographic inks and varnishes used in food packaging, reducing the amount of reactive diluents are known to reduce significantly migration and off-odor problems.

The present invention therefore relates to urethane(meth)acrylates according to formula (XVII) and (XVIII)

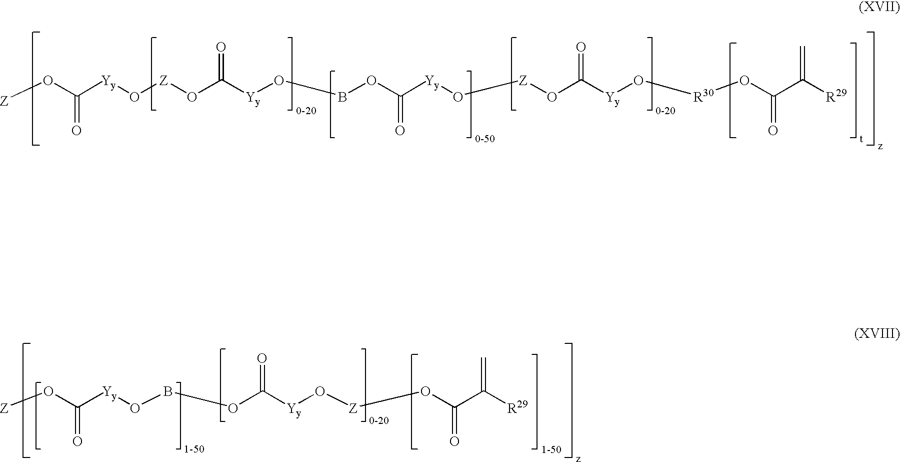

wherein:

Z is the residue of the hydroxyalkyl carbamate of formula (I), (II), (III), (IV), (V), (VI) and/or (VII);

z is an integer from 1 to the number of OH groups present in the hydroxyalkyl carbamate of formula (I), (II), (III), (IV), (V), (VI) or (VII);

B is the residue of the polyol such as defined here above;

R^{30′} represents an alkyl, which may contain from 1 to 10 ether bridges group, from 1 to 10 —O—CO—O— bridges and/or from 1 to 10 —O—CO— bridges (R^{30′} being the residue of R³⁰ as defined here above);

R²⁹ and t are such as defined here above;

Y is

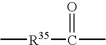

and y is 0 or 1.

The present invention more specifically relates to urethane(meth)acrylates according to formula (XVII) and (XVIII) wherein Z is the residue of hydroxyalkylcarbamates of formula (I), (II), (III), (IV), (V), (VI) and/or (VII) wherein R⁴, at least one of R⁵ and R⁶, at least one of R¹², R¹³ an R¹⁴, and at least one of R¹⁵ and R¹⁶ is different from hydrogen.

The present invention even more specifically relates to urethane(meth)acrylates according to formula (XVII) and (XVIII) wherein Z is the residue of hydroxyalkylcarbamates of formula (II) wherein R⁷ is ethylene and R⁵ and R⁶ together are ethylene.

The present invention also relates to urethane(meth)acrylates according to formula (XVII) and (XVIII) wherein Z is the residue of hydroxyalkylcarbamates of formula (II) wherein R⁷ is trimethylene, 2,2-dimethylpropylene, 1-methyltrimethylene, 1,2,3-trimethyltetramethylene, 2-methyl-pentamethylene, 2,2,4-(or 2,4,4-)trimethylhexamethylene, metaxylylene, cyclohexyl-1,3-ene, cyclohexyl-1,4-ene, 1,4-bis(propoxyl-3-ene)butane, N,N-bis(trimethylene)methylamine, 3,6-dioxaoctylene, 3,8-dioxadodecylene, 4,7,10-trioxatridecylene, poly(oxytetramethylene), poly(oxypropylene) with 2 to 15 1,2-propylene oxide units, poly(oxypropylene-co-oxyethylene) with 2 to 15 propylene oxide and 2 to 15 ethylene oxide units.

The present invention particularly relates to the urethane(meth)acrylates presented in the following examples.

### EXAMPLE 1

Step 1: Preparation of diurethanediol

100 parts of 2-methyl-1,5-pentanediamine (Dytek A, DuPont) was charged in a double-wall glass reactor with a capacity of 2 l sparged with nitrogen. The reactor was fitted with an agitator, a thermometer, a gas inlet tube, a double-wall glass addition funnel, a connection to vacuum and a vacuum-jacketed distillation column. A liquid semi-automatic splitter using a solenoid-activated PTFE valve and timer was used to control the reflux and takeoff from the distillation column into a cooled double-walled receiving flask. The splitter and a double-walled condenser connected to a cooling unit. 143 parts of ethylenecarbonate (Jeffsol E C, Huntsman) is slowly added while keeping the reactor under an inert atmosphere. Addition rate was chosen such as that the temperature in the reactor did not exceed 60° C.

After addition the temperature is raised to 70° C. until the reaction is complete, as indicated through titration of residual amine.

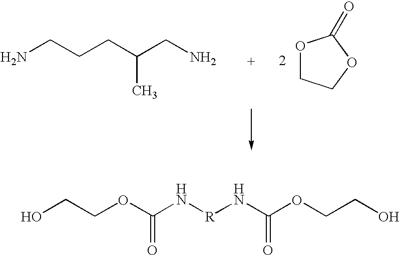

Step 2

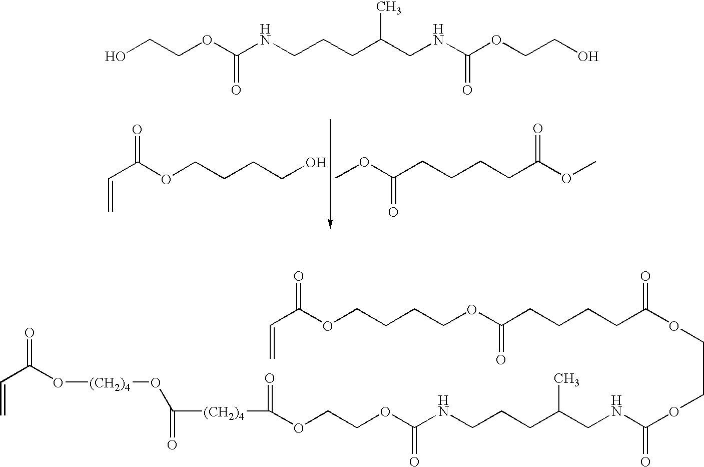

Then 289 parts of dimethyladipate, 241 parts of 4-hydroxybutylacrylate, 0.36 parts of methylhydroquinone and 250 parts of toluene are added to 40 parts to the product obtained in Step 1.

The mixture is heated to reflux and dried by azeotropic distillation, At this stage, air was injected throughout the reaction mixture. After adding 40 parts Tyzor TPT (tetrabutoxytitanate, produced by DuPont) the reaction mixture was maintained at 110-115° C. and the methanol generated was taken off overhead as a methanol/toluene azeotrope. The reaction was continued until a measure of the refractive index indicated that no more methanol was present in the distillate. Reaction time was 2.5 hours.

The reaction mixture was cooled down to 80° C., 120 parts of water was added, the mixture stirred for 2 hours and filtrated to remove the catalyst.

The filtrate is stripped under slight vacuum to remove the solvent and residual water and a clear, slightly yellow urethaneacrylate (2 Gardner) is obtained which is low viscous (4900 mPa·s at 25° C.).

### EXAMPLE 2

The first step of example 1 was repeated, except that the diamine was 1,3-(bisaminomethyl)cyclohexane

Step 2

Then 70 parts of dimethyladipate, 140 parts of polyadipate of 1,4-butanediol (molecular weight: 700) and 344 parts of toluene are added to 128 parts to the hydroxycarbamate obtained in Step 1.

The mixture is heated to reflux and dried by azeotropic distillation, At this stage, air was injected throughout the reaction mixture. After adding 4 parts Tyzor NBZ (tetrabutoxyzirconate, produced by DuPont) and 4 parts zirconiumacetylacetone (produced by Sachem Europe) the reaction mixture was maintained at 110-115° C. and the methanol generated was taken off overhead as a methanol/toluene azeotrope. The reaction was continued until a measure of the refractive index indicated that no more methanol was present in the distillate. After cooling the reaction mixture to 80° C., 344 parts of ethylacrylate and 0.36 parts of methylhydroquinone were added. The reaction mixture was heated to 110-115° C. and the ethanol generated was taken off overhead as a ethanol/toluene/ethylacrylate azeotrope. The reaction was continued until no more ethanol was present in the distillate.

The reaction mixture was cooled down to 80° C., 120 parts of water was added, the mixture stirred for 30 minutes and filtrated to remove the catalyst.

The filtrate is stripped under slight vacuum to remove the solvent and residual water and a clear, slightly yellow urethaneacrylate is obtained which is low viscous and has a hydroxyl value of less then 10 mg KOH/gram)

### EXAMPLE 3

The first step of example 1 was repeated.

Step 2

Then 139.4 parts of dimethyladipate, 119.4 parts of polyethyleneglycol (molecular weight: 600), 57.7 parts of 4-hydroxybutylacrylate, 0.1 parts of methylhydroquinone and 250 parts of toluene are added to 117 parts to the hydroxycarbamate obtained in Step 1.

The mixture is heated to reflux and dried by azeotropic distillation, At this stage, air was injected throughout the reaction mixture. After adding 7.67 parts Tyzor NBZ (tetrabutoxyzirconate, produced by DuPont) and 7.8 parts zirconiumacetylacetone (produced by Sachem Europe) the reaction mixture was maintained at 110-115° C. and the methanol generated was taken off overhead as a methanol/toluene azeotrope. The reaction was continued until a measure of the refractive index of the distillate indicated that no methanol was formed anymore.

The reaction mixture was cooled down to 80° C., 42 parts of water was added, the mixture stirred for 2 hours and filtered to remove the catalyst.

The filtrate is stripped under slight vacuum to remove the solvent and residual water and a slightly yellow urethaneacrylate is obtained which is low viscous and soluble in water.

### EXAMPLE 4

The first step of example 1 was repeated.

Step 2

Then 193.7 parts of diethylcarbonate, 114 parts of 1,6-hexanediol, 64.9 parts of 4-hydroxybutylacrylate, 0.1 parts of methylhydroquinone and 250 parts of toluene are added to 131.5 parts to the hydroxycarbamate obtained in Step 1.

The mixture is heated to reflux and dried by azeotropic distillation, At this stage, air was injected throughout the reaction mixture. After adding 15.7 parts Tyzor NBZ (tetrabutoxyzirconate, produced by DuPont) and 16 parts zirconiumacetylacetone (produced by Sachem Europe) the reaction mixture was maintained at 110-115° C. and the ethanol generated was taken off overhead as a ethanol/toluene azeotrope. The reaction was continued until a measure of the refractive index indicated that no more ethanol was present in the distillate

The reaction mixture was cooled down to 80° C., 42 parts of water was added, the mixture stirred for 30 minutes and filtered to remove the catalyst.

The filtrate is stripped under slight vacuum to remove the solvent and residual water and a slightly yellow urethaneacrylate is obtained which is low viscous.

### EXAMPLE 5

Step 1: Preparation of diurethanediol

100 parts of piperazine (Akzo Nobel) was charged in a double-wall glass reactor with a capacity of 2 l sparged with nitrogen. The reactor was fitted with an agitator, a thermometer, a gas inlet tube, a double-wall glass addition funnel, a connection to vacuum and an vacuum-jacketed distillation column. A liquid semi-automatic splitter using a solenoid-activated PTFE valve and timer was used to control the reflux and takeoff from the distillation column into a cooled double-walled receiving flask. The splitter and a double-walled condenser connected to a cooling unit. 205 parts of ethylenecarbonate (Jeffsol E C, Huntsman) is slowly added while keeping the reactor under an inert atmosphere. Addition rate was chosen such as that the temperature in the reactor did not exceed 80° C.

After addition the temperature is raised to 90° C. until the reaction is complete, as indicated through titration of residual amine.

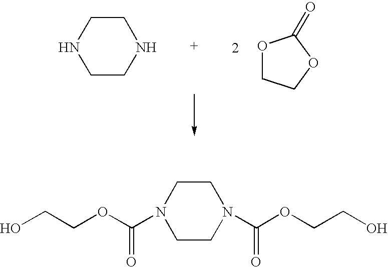

Step 2

Then 405 parts of dimethyladipate, 335 parts of 4-hydroxybutylacrylate, 0.5 parts of methylhydroquinone and 250 parts of toluene and 40 parts are added.

The mixture is heated to reflux and dried by azeotropic distillation, At this stage, air was injected throughout the reaction mixture. After adding 15 parts tetrabutoxyzirconate (Tyzor NBZ, produced by DuPont) and 15 parts zirconiumacetylacetone (produced by Sachem) the reaction mixture was maintained at 110-115° C. and the methanol generated was taken off overhead as a methanol/toluene azeotrope. The reaction was continued until a measure of the refractive index indicated that no more methanol was present in the distillate.

The reaction mixture was cooled down to 80° C., the catalyst hydrolyzed by adding 120 parts of a 10% phosphoric aqueous solution (w/w), the mixture stirred for 30 minutes at 80° C. and filtrated to remove the hydrolyzed catalyst.

The filtrate is stripped under vacuum to remove the solvent and residual water and a slightly yellow urethaneacrylate is obtained which is very low viscous (H, 2223 mPa·s at 25° C.)

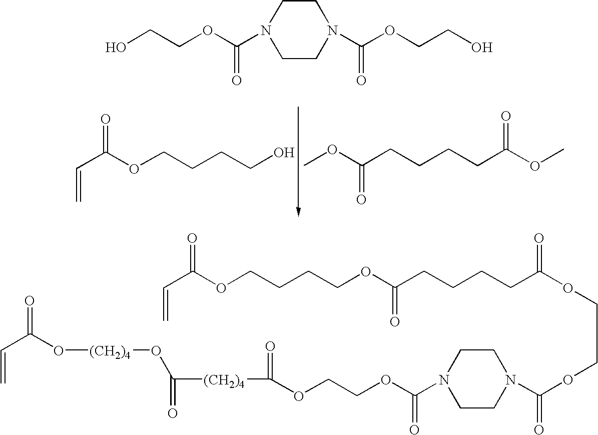

1. Process for producing urethane(meth)acrylates which comprises the reaction of (a) at least one hydroxyalkyl carbamate of formula (I), (II), (III), (IV), (V), (VI) or (VII) 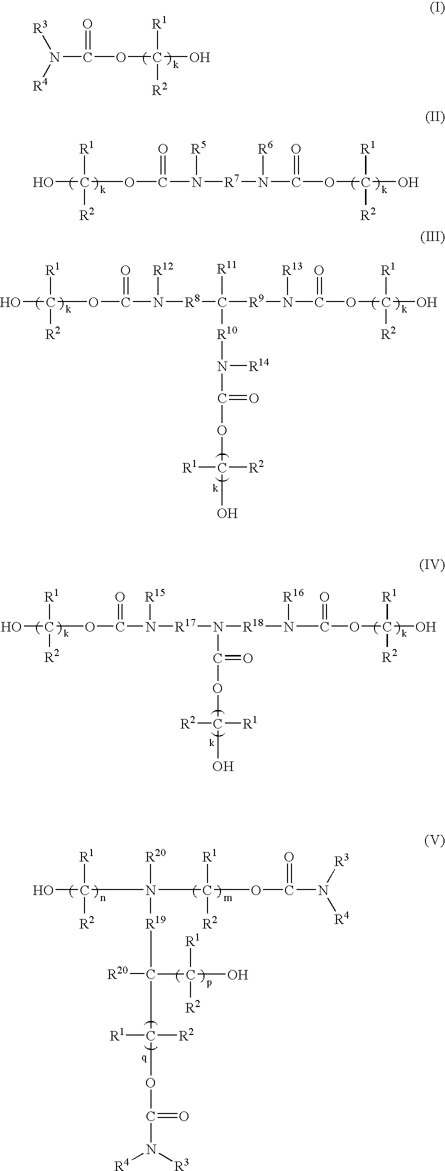 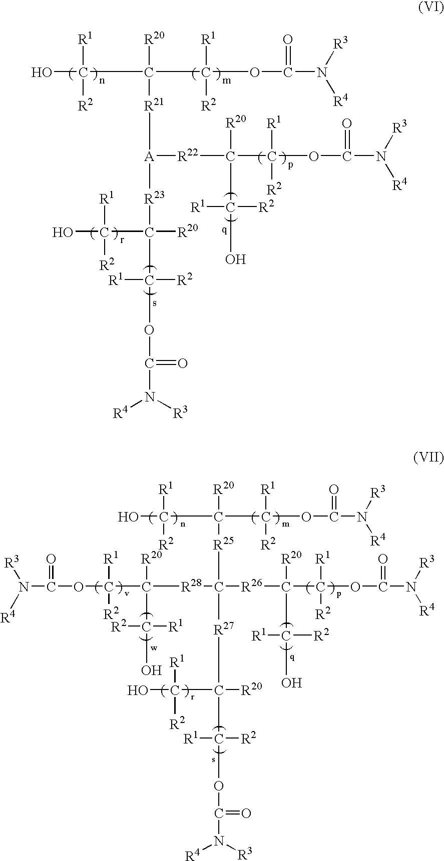
wherein
k≧2
n=0 to 2
m=0 to 2
n+m≧1
p=n or m, q=n or m, r=n or m, s=n or m, v=n or m, w=n or m
(p+q)=(r+s)=(v+w)=(n+m)
each R¹, each R², each R²⁰ is, independently, chosen from the group of
hydrogen,
halogen,
hydroxy,
alkyl, optionally substituted by hydroxy; halogen; aryl and/or aryl substituted by hydroxy, halogen or alkyl; and optionally containing from 1 to 8 ether bridges,
alkenyl, optionally substituted by hydroxy; halogen; aryl and/or aryl substituted by hydroxy, halogen or alkyl; and optionally containing from 1 to 8 ether bridges,
aryl, optionally substituted by hydroxy; halogen; alkyl; alkyl substituted by hydroxy, halogen and/or aryl; and/or alkyl containing from 1 to 8 ether bridges,
R³ is an alkyl, optionally substituted by hydroxy, tertiary amine and/or aryl, and optionally containing from 1 to 20 ether bridges and/or from 1 to 3 tertiary amine bridges,
R⁴, R⁵, R⁶, R¹², R¹³, R¹⁴, R¹⁵ and R¹⁶ are, independently, chosen from the group of
hydrogen, and
alkyl, optionally substituted by hydroxy, tertiary amine and/or aryl, and optionally containing from 1 to 8 ether bridges and/or from 1 to 3 tertiary amine bridges,
with the proviso that, respectively, R³ and R⁴, R⁵ and R⁶, R¹² and/or R¹³ and/or R¹⁴, R¹⁵ and R¹⁶ may be linked together in order to form a ring,
R⁷, R⁸, R⁹, R¹⁰, R¹⁷ and R¹⁸ are, independently, chosen from alkylene, alkenylene, arylene and aralkylene chains which may contain from 1 to 8 ether bridges and/or from 1 to 3 tertiary amine bridges,
R¹¹ is hydrogen or alkyl;
R¹⁹, R²¹, R²², R²³, R²⁵, R²⁶, R²⁷ and R²⁸, are, independently, chosen from alkylene, alkenylene, arylene and aralkylene chains which may contain from 1 to 20 ether bridges, from 1 to 4 tertiary amine bridges, from 1 to 4 —CO— bridges and/or from 1 to 4 —O—CO— bridges;
A is 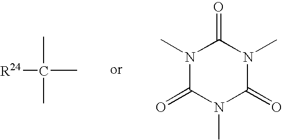
wherein R²⁴ is hydrogen or alkyl;
(b) at least an (meth)acrylate of formula (VIII)
[CH₂═CR²⁹—CO—O—]ₜ—R³⁰ (VIII)
wherein R²⁹ is hydrogen or methyl, and R³⁰ represents an alkyl, optionally substituted by hydroxy, which may contain from 1 to 10 ether bridges group, from 1 to 10 —O—CO—O— bridges and/or from 1 to 10 —O—CO— bridges; t≧1; and
(c) at least one carbonate of formula (IX) and/or a diester of formula (X) 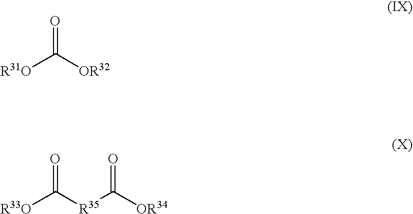
wherein
each R³¹, each R³², each R³³, each R³⁴ is, independently, chosen from the group of alkyl and aryl, R³⁵ is alkylene, alkenylene or arylene; and
(d) optionally, at least one polyol different from the hydroxyalkyl carbamates (a);
in the presence of at least one transesterification catalyst.

2. The process according to claim 1, wherein the hydroxyalkyl carbamates of formula (I), (II), (III) and (IV) are obtained by reacting amines of, respectively, formula (IX), (X), (XI) and (XII) 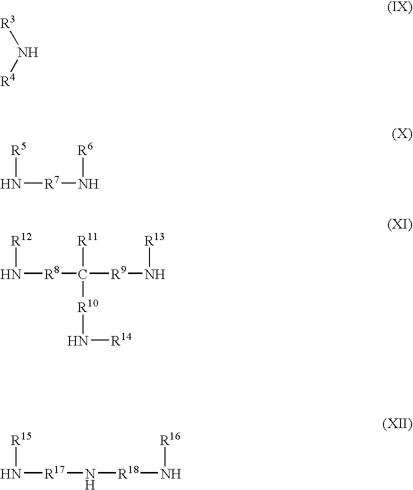
wherein R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸, are defined as in claim 1, with a cyclic carbonate of formula (XIII) 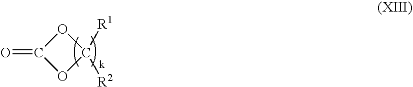
wherein R¹, R² and k are defined as in claim 1.

3. The process according to claim 1, wherein the hydroxyalkyl carbamates of formula (V), (VI) and (VII) are obtained by reacting an amine of formula (IX) 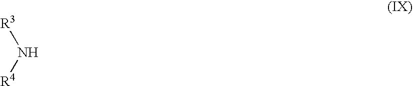
wherein R³ and R⁴ are defined as in claim 1, with, respectively, a cyclic carbonate of formula (XIV), (XV) and (XVI) 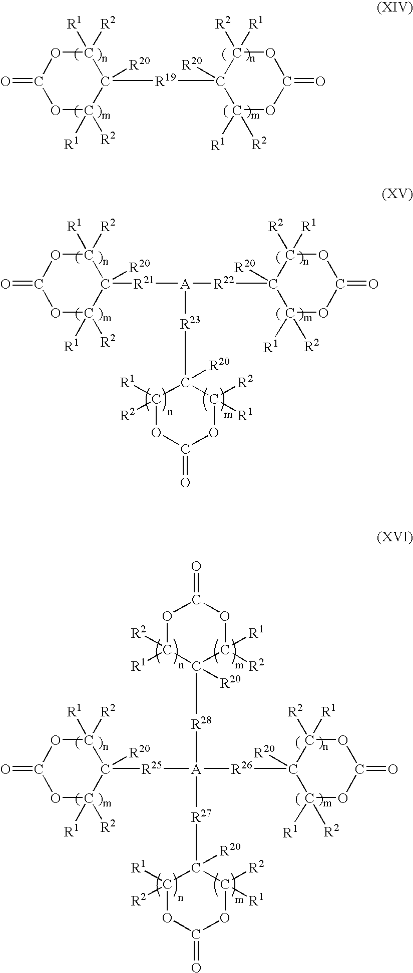
wherein R¹, R², R¹⁹, R²⁰, R²¹, R²², R²³, R²⁴, R²⁵, R²⁶, R²⁷, R²⁸, A, n and m are defined as in claim 1.

4. The process according to claim 1, wherein the transesterification catalyst is selected from organotitanates, organozirconates and organotin catalysts.

5. The process according to 4, wherein the transesterification catalyst is an alkyltitanate wherein each alkyl, independently, comprises from 2 to 8 carbon atoms or an alkylzirconate wherein each alkyl, independently, comprises from 2 to 8 carbon atoms or a zirconium 1,3-diketone chelate or a mixture thereof.

6. The process according to claim 1, wherein the transesterification reaction is conducted in the presence of at least one polymerization inhibitior.

7. The process according to claim 1, wherein the temperature during the transesterifcation reaction is at most 120° C.

8. The process according to claim 1, wherein the weight ratio of catalyst to the generated urethane(meth)acrylate is from 0.001 to 0.2.

9. The process according to claim 1, wherein k=2 or 3 and n+m, p+q, r+s, v+w=1.

10. The process according to claim 1, wherein in formula (I), (II), (III) and (IV) one of the R¹ substituents is chosen from the group of hydrogen, methyl, ethyl, hydroxymethyl, chloromethyl, allyloxymethyl, the R² substituent present on the same substituent as said R¹ subtituent is chosen from hydrogen and methyl, and all other R¹ and R² substituents are hydrogen.

11. The process according to claim 1, wherein in formula (V), (VI) and (VII) each R¹, each R² and each R²⁰ is hydrogen.

12. The process according to claim 1, wherein in formula (I), (V), (VI) and (VII) and (IX) R⁴ is hydrogen and R³ is an alkyl comprising at least 3 carbon atoms and substituted by at least one hydroxy and optionally containing one or two ether bridges.

13. The process according to claim 1, wherein in formula (VIII) t is 1 and wherein R³⁰ is an alkyl comprising from 1 to 6 carbon atoms or an alkyl substituted by at least one hydroxy group, and which may contain from 1 to 10 ether bridges group, from 1 to 10 —O—CO—O— bridges or from 1 to 10 —O—CO— bridges.

14. The process according to claim 1, wherein in the carbonates of formula (IX) R³¹ and R³² are chosen from the group of alkyl comprising form 1 to 4 carbon atoms and from phenyl.

15. The process according to claim 1, wherein in the diesters of formula (X) R³³ and R³⁴ are chosen from the group of alkyl comprising form 1 to 6 carbon atoms and from phenyl, and wherein R³⁵ is an alkylene or alkenylene comprising from 1 to 10 carbon atoms or phenylene

16. The process according to claim 1, wherein the polyol (d) responds to formula
B—(OH)ₓ wherein x is an integer from 1 to 6 and B represents an alkyl or alkenyl optionally containing from 1 to 100 ether bridges, —CO—O— bridges, —CO— bridges and/or —O—CO—O— bridges and/or containing one or more —COOH, —SO₃H and/or —PO₄H groups.

17. The process according to claim 16, wherein the polyol (d) is chosen from ethylene glycol, propyleneglycol, 1,4-butanediol, 1,5-pentanediol, neopentylglycol, 1,6-hexanediol, diethyleneglycol, triethyleneglycol, dipropyleneglycol, tripropyleneglycol, cyclohexanedimethanol, dimethylolpropionic acid, trimethylolpropane, pentaerythritol and macrodiols such as polyetherdiols, polyesterdiols, polycarbonatediols, polyestercarbonatediols, polybutadienediol, acrylic diols, and their mixtures.

18. The process according to claim 1, wherein the equivalent ratio of (meth)acrylate of formula (VIII) to hydroxyalkyl carbamate is from 0.01 to 7.

19. The process according to claim 1, wherein the equivalent ratio of carbonate (IX) and/or diester (X) to hydroxyalkyl carbamate is from 0.05 to 10.

20. The process according to claim 1, wherein the equivalent ratio of polyol (d) to hydroxyalkyl carbamate is from 0 to 50.

21. Urethane(meth)acrylates obtainable by the process according to claim 1.

22. Urethane(meth)acrylates responding to formula (XVII) and (XVIII) 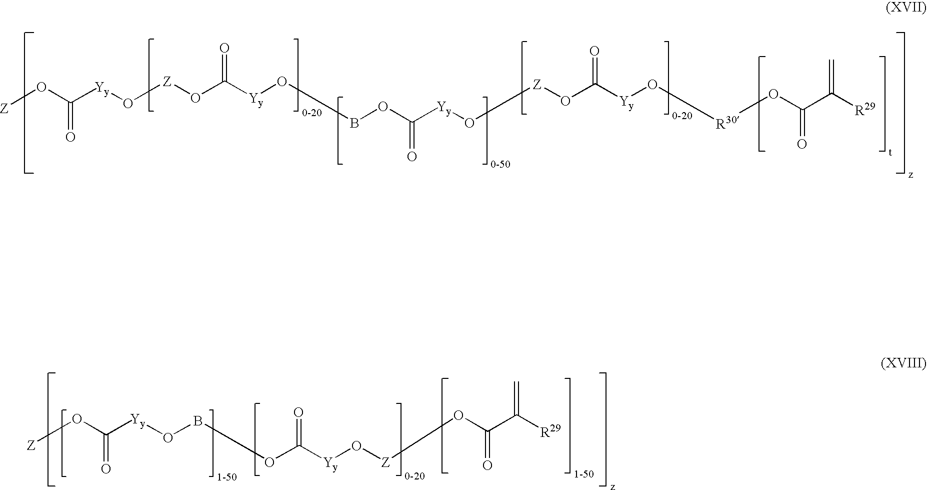
wherein
Z is the residue of the hydroxyalkyl carbamate of formula (I), (II), (III), (IV), (V), (VI) and/or (VII);
z is an integer from 1 to the number of OH groups present in the hydroxyalkyl carbamate of formula (I), (II), (III), (IV), (V), (VI) or (VII) such as defined in claim 1;
B is the residue of the polyol as defined in claim 16;
R^{30′} represents an alkyl, which may contain from 1 to 10 ether bridges group, from 1 to 10 —O—CO—O— bridges and/or from 1 to 10 —O—CO— bridges
R²⁹ and t are such as defined in claim 1;
Y is 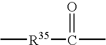
wherein R35 is defined in claim 1;
and y is 0 or 1.

23. Urethane(meth)acrylates according to claim 22, wherein Z is the residue of hydroxyalkylcarbamates of formula (I), (II), (III), (IV), (V), (VI) and/or (VII) wherein R⁴, at least one of R⁵ and R⁶, at least one of R¹², R¹³ an R¹⁴, and at least one of R¹⁵ and R¹⁶ is different from hydrogen.

24. Urethane(meth)acrylates according to claim 22, wherein Z is the residue of hydroxyalkylcarbamates of formula (II) wherein R⁷ is ethylene and R⁵ and R⁶ together are ethylene.

25. Urethane(meth)acrylates according to claim 22, wherein Z is the residue of hydroxyalkylcarbamates of formula (II) wherein R⁷ is trimethylene, 2,2-dimethylpropylene, 1-methyltrimethylene, 1,2,3-trimethyltetramethylene, 2-methyl-pentamethylene, 2,2,4-(or 2,4,4-)trimethylhexamethylene, metaxylylene, cyclohexyl-1,3-ene, cyclohexyl-1,4-ene, 1,4-bis(propoxyl-3-ene)butane, N,N-bis(trimethylene)methylamine, 3,6-dioxaoctylene, 3,8-dioxadodecylene, 4,7,10-trioxatridecylene, poly(oxytetramethylene), poly(oxypropylene) with 2 to 15 1,2-propylene oxide units, poly(oxypropylene-co-oxyethylene) with 2 to 15 propylene oxide and 2 to 15 ethylene oxide units.

26. Method of preparing a radiation curable composition which comprises employing the urethane(meth)acrylate of claim 1 therein.
